(19) United States
(12) Patent Application Publication
(10) Pub. No.: US 2013/0341424 A1
(43) Pub. Date: Dec. 26, 2013

(54) AIR FRESHENER VENT CLIP AND ACCESSORY

(71) Applicant: Hayloft Enterprises, Inc., Wilmington, DE (US)

(72) Inventors: Kyle Brandenburg, Middletown, DE (US); Frank M. Lesniak, Media, PA (US)

(73) Assignee: Hayloft Enterprises, Inc., Wilmington, DE (US)

(21) Appl. No.: 14/011,849

(22) Filed: Aug. 28, 2013

Related U.S. Application Data
(63) Continuation of application No. 12/617,035, filed on Nov. 12, 2009.

(51) Int. Cl. A61L 9/12

(52) U.S. Cl. CPC A61L 9/12
USPC 239/55; 239/57

(57) ABSTRACT

An air freshener clip removably engages with a louver or slat of a vent, such as an automotive heating and air conditioner vent. The clip may have a curved body that tapers from a clip bend at its proximal end to a tip at its distal end. When the clip is engaged over the louver or slat, the distal end of the curved body extends into the vent opening. The air freshener clip has an outer portion formed from one or more fragrance loaded polymers, and releases fragrance into an air stream that contacts the outer portion on the curved body and is directed out of the vent. An accessory may be removably attached or link to the clip to decorate or adorn the clip.

### CROSS-REFERENCE TO RELATED APPLICATION

This application is a continuation of U.S. patent application Ser. No. 12/617,035, filed Nov. 12, 2009, now pending.

### FIELD OF THE INVENTION

The present invention relates to a curved clip formed of a polymer resin and having at least an outer portion loaded with a fragrance oil or oils or other fragrance material, where such clip is adapted to be held directly in the flow of air through a vent, such as an automotive forced air vent, to release fragrance to such air flow. The present invention further relates to an accessory that is removably linked to the clip to decorate or adorn the clip.

### BACKGROUND OF THE INVENTION

The passenger compartments of automobiles and trucks may retain unpleasant odors introduced into such compartment by drivers, passengers or cargo. Examples of unpleasant odors include cigarette and cigar smoke, food odors, body odors, retained cargo odors and gas or exhaust fumes. Various air freshener devices have been designed for use in vehicle passenger compartments, including devices that suspend from a rear view mirror or from a sun visor. Such devices release fragrance into the air, but lack a source of forced air to enhance the rate of fragrance release,

U.S. Pat. No. 4,813,344 shows a deodorizer attached to a louver or slat of a heating or air conditioner vent. Attachment is by hook and loop fasteners (Velcro tape). When in use, a cover is pulled away from the vent and further into the passenger compartment to expose deodorant held within a compartment of the device. No portion of the deodorizer extends into the vent opening beyond the louver to which it is adhesively attached.

U.S. Pat. Nos. 6,264,887 and 6,123,906 disclose an air freshening device formed as a clip with two spaced apart straight legs that fit over a louver or slat of a heating or air conditioner vent so that the distal ends of the legs extend into the vent opening. Each of the straight legs has through holes therein for increasing air contact through the legs where fragrance is introduced. The legs terminate at a proximal end at a curved hand grasp that extends outwardly from the vent opening a substantial distance.

U.S. Pat. No. 5,407,642 shows a clip mount air freshener assembly in which the air freshener element is inserted into a straight holder that may be rotated into various orientations. The holder is attached to a clip that clips over a louver or slat of a heating or air conditioner vent. At all times, the air freshener element is held outside of the vent opening and air exiting that opening is forced against the air freshener element.

U.S. Pat. Nos. 5,527,493 and 5,478,505 show an air treating device that may be clipped to a louver or slat of a heating or air conditioner vent, or may be clamped to a sun visor. When clipped to a louver or slat, the air treating device remains in front of the vent opening.

U.S. Pat. No. 6,197,263 shows an automobile air freshener that is detachably connected with clamps to a louver or slat of a heating or air conditioner vent. The rectangular-shaped fragrance dispersing unit is heat activated.

U.S. D437,041 depicts an air freshener with a clip near one end.

Improvements to air freshener devices for use in conjunction with vent systems continue to be sought.

### SUMMARY OF THE INVENTION

In a first aspect, an air freshener has a curved body with a proximal end and a distal end. The curved body optionally tapers from a first width at its proximal end to a tip of a smaller second width at the distal end. Preferably the air freshener forms a clip having a clip arm with at least one bend therein, more preferably multiple bends therein, is formed at the proximal end of the curved body. An outer portion of polymer loaded with one or more fragrances is associated with or in contact with or over molded over at least a portion of the curved body. Preferably, the curved body has a concavely curved bottom surface with a radius of curvature in the range of about 1 cm to about 100 cm, more preferably about 5 to about 20 cm. Preferably, the distal end of the curved body is displaced from a plane defined by an upper exterior surface of the proximal end at an angle in the range of about 1 to about 30 degrees.

Preferably, the curved body is formed of a polymer such as polyvinylchloride, polyethylene, polyethylene terephthalate (PET), polyoxymethylene (Delrin), polyamide (nylon), low density polyethylene (LDPE), high density polyethylene (HDPE), polypropylene, ethylene vinyl acetate, acrylonitrile butadiene styrene (ABS), styrene-acrylonitrile (SAN), copolymers thereof, and mixtures thereof. The outer portion is formed of a polymer that may be the same as or different from the polymer forming the body. Preferably, the outer portion is formed of a polymer such as polyvinylchloride, polyethylene, low density polyethylene (LDPE), high density polyethylene (HDPE), thermoplastic elastomer (TPE), polypropylene, ethylene vinyl acetate, ELVAX® EVA ethylene vinyl acetate copolymer, acetate, butyrate, propionate, copolymers thereof, and mixtures thereof. At least the polymer forming the outer portion is loaded with a fragrance material, such as one or more natural and synthetic fragrance oils.

In another aspect, the air freshener body defines a plane at an upper exterior surface at its proximal end and the distal end of the body flexes or bends from a first position to a second position at an angle of from about 1 to about 30 degrees from the plane.

In yet another aspect, the outer portion is a jacket that is over molded over the body.

In still another aspect, an accessory is removably linked to at least one bend or at least one portion of the clip arm. Preferably, at least one projection extends from the accessory, and the projection(s) removably clip or fasten the accessory to the at least one bend or to at least one portion of the clip arm of the air freshener. The accessory may include artwork or advertising indicia on its front face.

### DESCRIPTION OF THE FIGURES

Numerous other objects, features and advantages of the invention shall become apparent upon reading the following detailed description taken in conjunction with the accompanying drawings, in which:

FIG. 1 is a right front perspective view of an air freshener clip according to the invention;

FIG. 2 is a left bottom perspective view of the air freshener clip of FIG. 1;

FIG. 3 is a cross-sectional view taken along line 3-3 in FIG. 1;

FIG. 4 is a cross-sectional view taken along line 4-4 in FIG. 1;

FIG. 5 is a left front perspective view in partial phantom outline of the air freshener clip of FIG. 1 installed within an automotive heating and air conditioning discharge vent;

FIG. 6 is left side elevational view of the air freshener clip of FIG. 1 as installed within an automotive heating and air conditioning discharge vent;

FIG. 7 is a right front perspective view of an accessory plate;

FIG. 8 is a left rear perspective view of the accessory plate of FIG. 7;

FIG. 9 is a left rear perspective view of the accessory plate of FIG. 7 joined to the air freshener clip of FIG. 1; and

FIG. 10 is a left side elevational view of the air freshener clip of FIG. 1 with accessory plate of FIG. 7 joined thereto, as installed within an automotive heating and air conditioning discharge vent.

### DESCRIPTION OF THE PREFERRED EMBODIMENTS

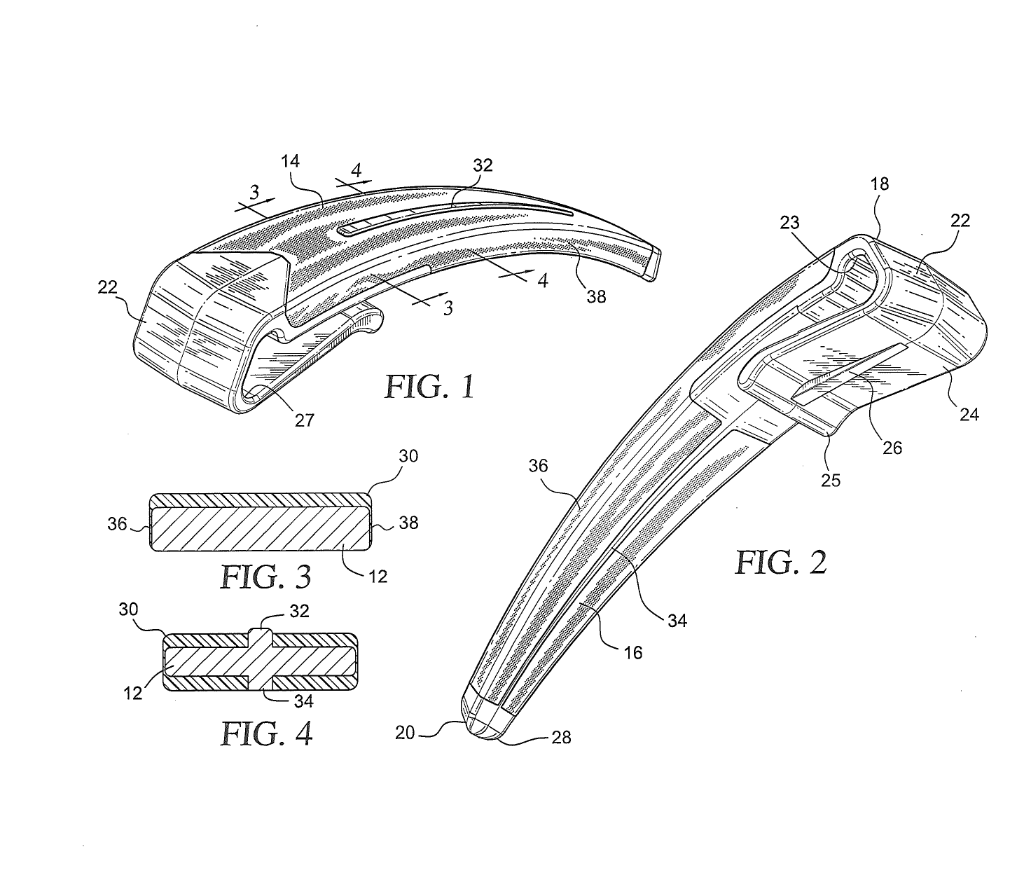

With reference to the drawings in which like numerals designate similar elements, an air freshener clip 10 is shown in FIGS. 1-4. The air freshener clip 10 is formed of a body or base or frame 12 having a top surface 14 and a bottom surface 16. The body 12 has a proximal end 18 and a distal end 20. In one embodiment, the body 12 has a length in the range of 6 to 10 cm as measured from its proximal end 18 to its distal end 20. The body 12 curves concavely between the proximal end 18 and the distal end 20. The body 12 shown in FIGS. 1-4 also tapers from a first width at its proximal end 18 to a second width at its distal end 20, wherein the body 12 is wider at the proximal end 18 and forms a narrower tip 28 at its distal end 20. In the embodiment shown in FIGS. 1-4, the width at the proximal end 18 preferably is between about 1 and 2 cm and the width at the distal end 20 preferably is between about 0.5 and 1 cm.

A clip bend 22 at the proximal end 18 joins clip arm 24 to the proximal end 18 of the body 12. The clip arm 24 terminates at an end 25 of the clip arm. Together, the clip arm 24 and clip bend 22 generally form a U-shape in conjunction with the bottom surface 18. Optionally, clip arm 24 is reinforced by one or more reinforcing ribs 26.

An upper ridge 32 optionally may extend outwardly from the top surface 14 of the body 12. A lower ridge 34 optionally may extend outwardly from the bottom surface 16 of the body 12.

An outer portion 30, which may be a jacket, is attached, formed or molded over at least a portion of the left side 36 and right side 38 surfaces of the body 12. Preferably, the outer portion 30 also is formed or molded over top surface 14 and bottom surface 16 of the body 12. As shown in FIGS. 3 and 4, the outer portion 30 may be molded over a portion of the top surface 14, the bottom surface 16 and sides 36, 38 of the body 12. Preferably, for aesthetics and better over molding registration, the outer portion 30 is a jacket that is over molded over the curved body and anchored to at least one of the upper ridge 32 or lower ridge 34. In the embodiment shown in FIGS. 1-4, the outer portion 30 is not molded over the upper ridge 32 and lower ridge 34 of the body 12.

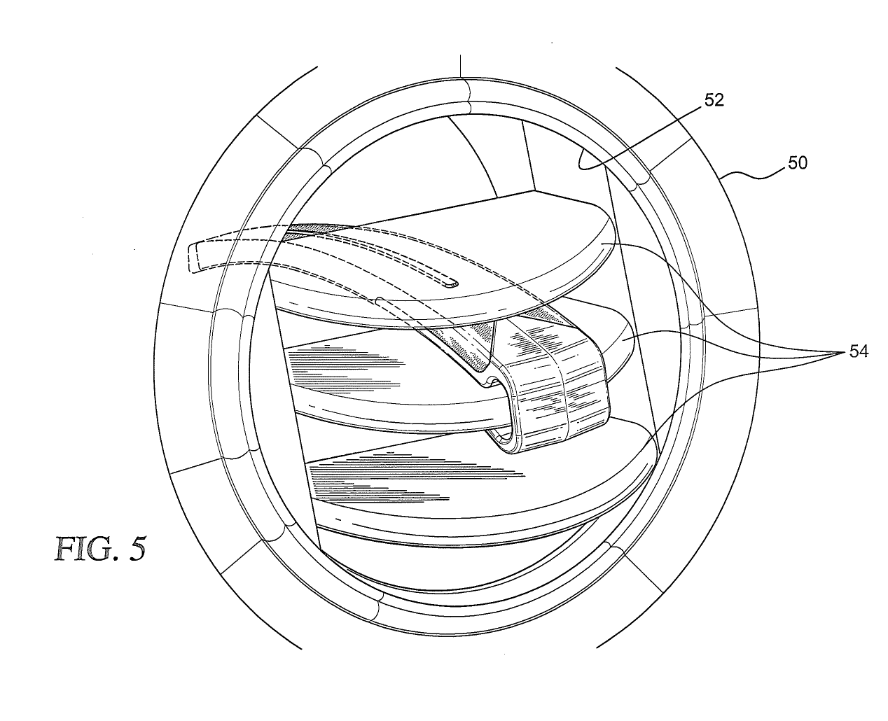

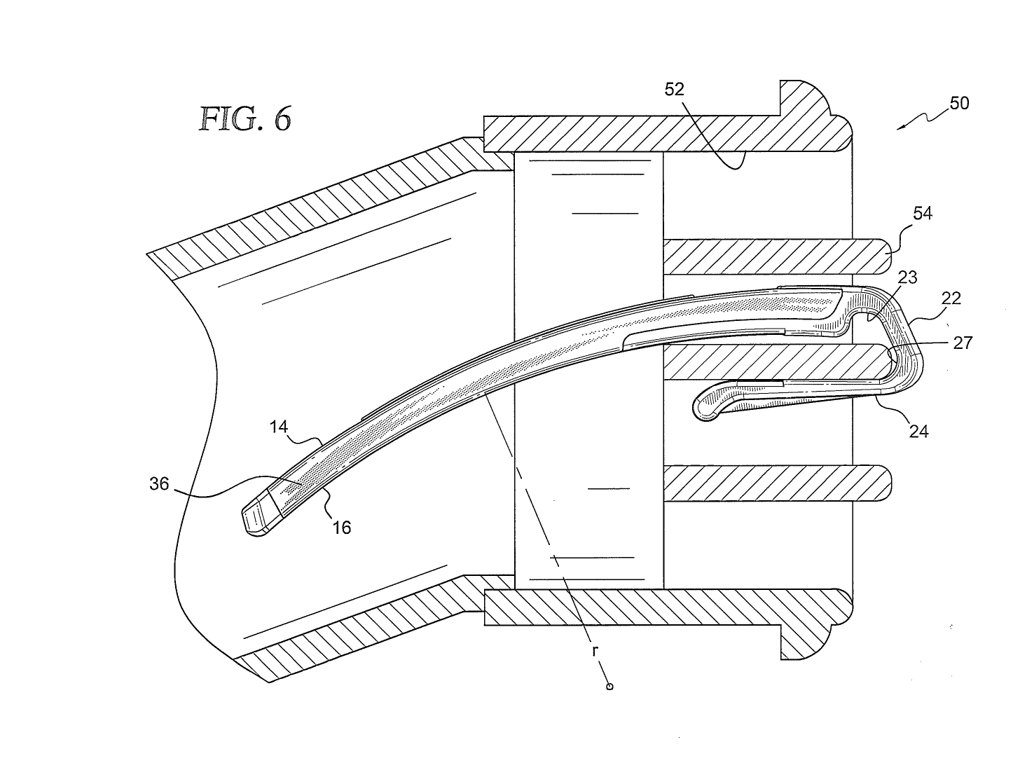

Referring next to FIGS. 5 and 6, the air freshener clip 10 is mounted to a louver or slat 54 in a vent opening 52 of an automotive heating and air conditioning air grill or vent 50. The clip arm 24 resiliently flexes open to engage the louver 54 and then closes to hold the air freshener clip 10 within the vent opening 52. The proximal end 18 of the air freshener clip 10 remains accessible to the vehicle occupant, so that the air freshener clip 10 may be disengaged when desired by pulling clip arm 24 and clip bend 22 off of the louver 54. When the air freshener clip 10 is installed and in use, the distal end 20 of the air freshener clip 10 extends a distance into the vent opening 52.

The air freshener clip 10 shown in FIGS. 5-6 has been installed in vent opening 52 that has a contour sufficient to receive the entire length of the air freshener clip 10 without deforming the air freshener clip 10. In some instances, however, vent openings or other passageways or constrictions into which an air freshener clip 10 is to be installed have more severely bent sidewalls. The curved and tapered configuration of air freshener clip 10 permits at least a portion of the body 12, for example, the distal end 20, to be bent upwardly or downwardly with respect to a central axis of the body 12. Thus, the air freshener clip 10 may be installed into more restrictive confines of vent openings or other air flow constrictions with a more severely bent contour than is shown in FIG. 6. Preferably, the distal end 20 can be flexed or bent up or down and/or side to side within an angular range of plus or minus 1 degree to 180 degrees from a central axis of the body 12, preferably within an angular range of plus or minus 1 degree to 90 degrees. Alternatively, the distal end 20 may be displaced from or flex from a plane defined by an upper exterior surface of the proximal end at an angle in the range of plus or minus about 1 to about 180 degrees, preferably plus or minus 1 degree to 90 degrees. In one embodiment, the clip body is sufficiently bendable to form it into a hook or “J” shape. Depending upon polymer(s) selected to form the air freshener clip 10, the clip 10 may have sufficient resilience to return to its unflexed shape when it is removed from restrictive confines.

Forced air emitted from the heating and air conditioning system is forced against the curved body 12 and jacket 30 of the air freshener clip 10. The concavely curved contour of the lower exterior surface of the curved body 12 creates a turbulent flow that catches and directs more air across the jacket surfaces, particularly the bottom and side surfaces. Fragrance material loaded in the jacket polymer material releases fragrance into the air that contacts the air freshener clip 10 and then enters into the passenger compartment of the motor vehicle. Preferably, the curved body 12 has a concavely curved bottom surface 16 with a radius of curvature r (see FIG. 6) in the range of about 1 cm to 100 cm, more preferably in the range of about 5 to about 20 cm.

As shown in FIG. 6, the clip bend 22 may be formed to have a first curved portion 23 and a second curved portion 27. When clip bend 22 is closed without being attached over a louver or slat, curved portion 23 may form an angle in the range of from 80 to 110 degrees between the top surface 14 and clip bend 22, and curved portion 27 may form an angle in the range of from 45 to 100 degrees between the clip arm 24 and clip bend 22.

In the embodiment shown in FIG. 6, a proximal end 17 of bottom surface 16 forms a first wall of curved portion 23. An inner surface of clip arm 24 forms a second wall of curved portion 23. The clip arm 22 has dual bends 23, 27 therein, which increases strength of the gripping action of clip arm 24. In addition, the bend of curved portion 23 results in an open space between the proximal end 17 and the inner surface of clip arm 24.

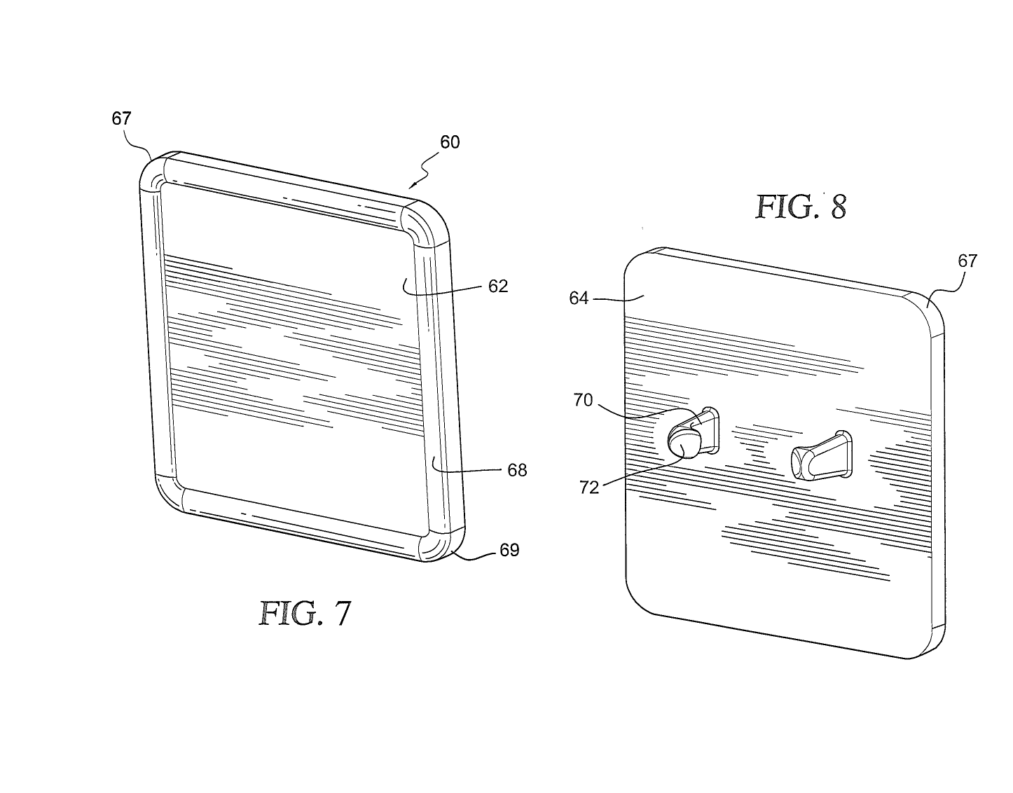

FIGS. 7 and 8 show an accessory plate or decorative element 60 that is adapted to be attached to an air freshener 10. The accessory plate or decorative element 60 has a front face 62 and a rear face 64. As shown in FIGS. 7 and 8, the accessory plate 60 has a generally square periphery with a side frame 68 having rounded corners 69. Alternative peripheries, such as oval, circular, triangular, irregular, etc., may be chosen as a matter of design choice, and the invention is not limited to accessory plates with generally square peripheries. A label or decal (not shown) may be adhered to the front face 62 surface of the decorative element. When used, the accessory plate 60 decorates or adorns the vent clip.

In FIG. 8, two projections or prongs 70 extend from a surface of the rear face 64 of the accessory plate or decorative element 60. As shown in FIG. 8, the prongs 70 are spaced apart from one another and have curved prong tips 72 each of which extends toward the opposite prong 70. An axis of rotation is defined between the opposite curved prong tips 72. Curved prong tips 72 releaseably snap fit or connect the accessory plate 60 to an air freshener clip 10. While two projections 70 are illustrated, the accessory plate or decorative element 60 may have one, two or more projections to facilitate attachment to the clip.

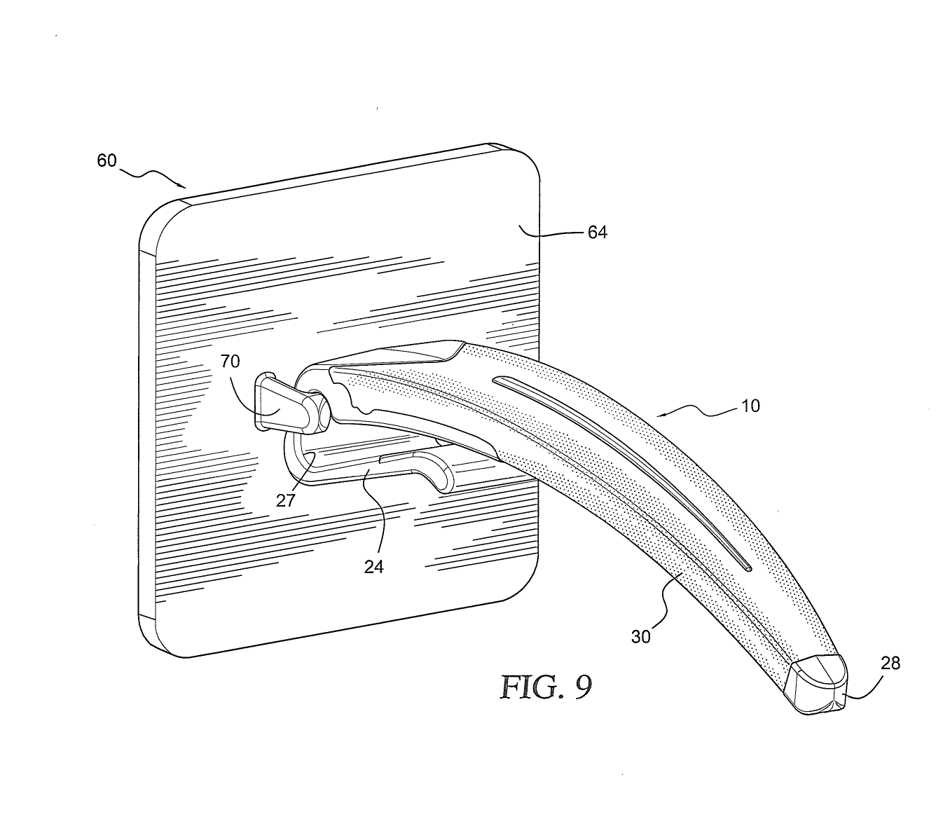

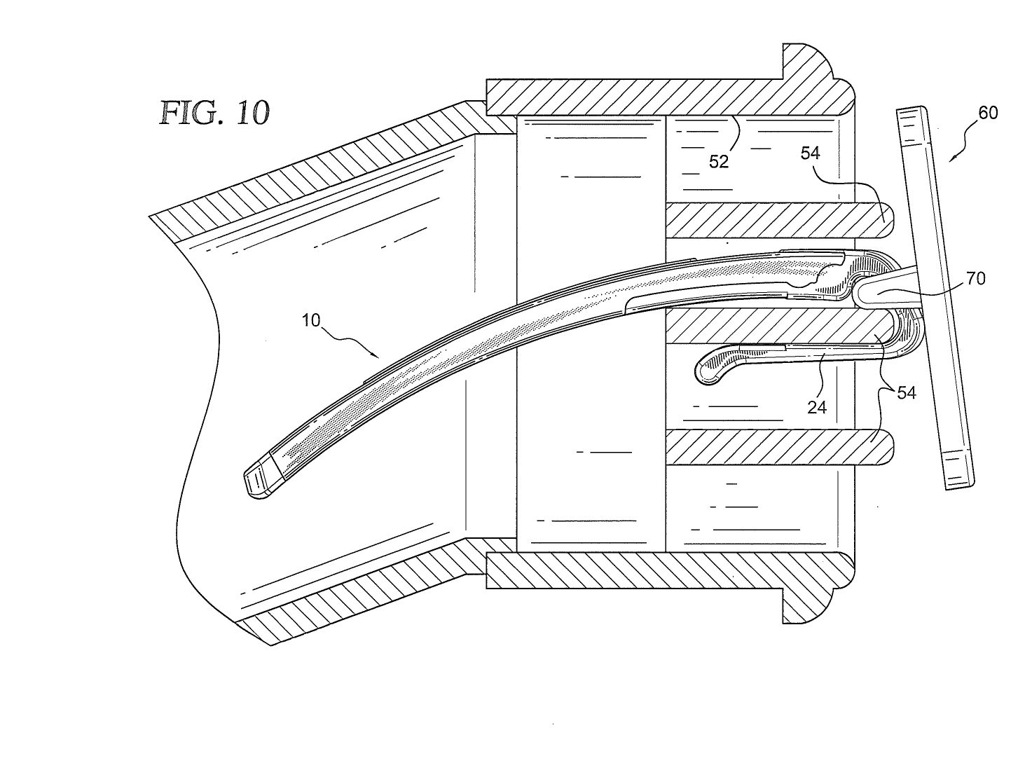

The accessory plate 60 is attached to the air freshener clip 10 in FIGS. 9 and 10. As shown, the curved prong tips 72 fit within the clip bend 22. With the accessory plate 60 engaged or joined thereto, the air freshener clip 10 still may be mounted over a louver or slat 54 in a vent opening 52 of an automotive heating and air conditioning air grill or vent 50. The accessory plate 60 may be rotated about the axis formed between curved prong tips 72 to adjust the position of the accessory plate front face 62 with respect to the passenger compartment of the vehicle and with respect to the vent opening 52. As shown in FIG. 10, the accessory plate 60 is positioned at an angle with respect to the clip bend 22 to face upwardly.

The accessory plate 60 may be mounted to the air freshener clip 10 before the air freshener 10 is mounted over a louver or slat 54 in a vent opening 52 of an automotive heating and air conditioning air grill or vent 50. The dual bend in the clip bend 22 of the clip arm 24 provides an ideal seating location for snap fitting the curved prong tips 72 of the accessory plate 60 to the air freshener 10. The accessory plate 60 may be released by exerting a pulling force to disconnect the curved prong tips 72 from the clip bend 22. Accordingly, different accessory plates 60 may be interchangably joined to and removed from the air freshener 10, either before the air freshener is installed or while the air freshener remains installed over a louver or slat 54. Alternatively, any form of accessory, including but not limited to accessory plates, may be joined to and removed from the air freshener clip 10 by releasable attachment to the curved portion 23 in clip bend 22.

The body 12 may be molded from a polymer containing at least one resin selected from the group consisting of: polyvinylchlorides (PVC), polyethylenes (PE), polyethylene terephthalates (PET), polyoxymethylene (Delrin), polyamides (nylon), low density polyethylene (LDPE), high density polyethylene (HDPE), polypropylenes, ethylene vinyl acetate, acrylonitrile butadiene styrene (ABS), and styrene-acrylonitrile (SAN), copolymers thereof and mixtures thereof. The resin composition may be mixed, blended or compounded together optionally with additives known in the art and with one or more fragrance materials to form an injection-moldable mixture before injection molding.

The outer portion 30 may be a different polymer resin from that selected for the body. The outer portion may be joined to the body 12 by adhesive or other attachment mechanism or may be in the form of a jacket that is over molded over the body 12 in a multiple shot molding process. In a multiple shot molding process, the body 12 is formed in a first molding step and the outer portion 30 or jacket is formed over the body 12 in a second or subsequent molding step.

The outer portion 30 may be molded from at least one resin selected from the group consisting of: polyvinylchlorides (PVC), polyethylenes (PE), high density polyethylenes (HDPE), low density polyethylene (LOPE), polyvinylchoride (PVC), polypropylenes, ethylene vinyl acetate copolymers (EVA), such as ELVAX® EVA ethylene vinyl acetate copolymer, thermoplastic elastomers (TPE), polypropylenes, acetate, butyrate and propionate, copolymers thereof, and mixtures thereof. The resin composition may be mixed, blended or compounded together with one or more fragrance materials and optionally with other additives known to those in the art to form an injection-moldable mixture before injection molding.

Most conventional fragrance materials are highly volatile essential oils. Even those which are less volatile contain highly volatile “top note” fractions which preferably are retained in the composition during processing to obtain desired fragrance power and olfactory impact by the resultant molded article. A fragrance material or a combination of fragrance materials that is compatible with the selected resin composition is preferred. The fragrance material may be a synthetically formed material or may be a naturally derived oil such as, but not limited to, the oil of Bergamot, Bitter Orange, Caraway, Cedar Leaf, Cedar Wood, Champacc, Cinnamon, Frankincense, Geranium, Lavendar, Mimosa, Orange, Orignaum, Patchouli, Rosewood, Sandalwood, Vanilla, Violet, White Cedar, Ylang Ylang, or the like. The particular essential oil or combination of oils to be used depends upon the particular fragrance desired for emission by the product formed.

Available fragrance materials are catalogued and described in references and databases known to persons skilled in the art. For example, a database is maintained by the Research Institute for Fragrance Materials at www.rifm.org. Fragrance material suppliers include Takasago International Corp. (Rockleigh, N.J.) and Symrise (Teterboro, N.J.). Exemplary synthetic fragrance materials are described in U.S. Pat. Nos. 4,411,829; 4,314,915 and 4,434,306.

The accessory plate or decorative element 60 may be molded from a polymer containing at least one resin selected from the group consisting of: polyethylenes (PE), high density polyethylenes (HDPE), low density polyethylene (LDPE), polyvinylchoride (PVC), polypropylenes, ethylene vinyl acetate copolymers (EVA), thermoplastic elastomers (TPE), acetate, butyrate and propionate, copolymers thereof, and mixtures thereof. Optionally, the resin composition may be mixed, blended or compounded together with one or more fragrance materials or other additives known to those in the art to form an injection-moldable mixture before injection molding.

The invention has been illustrated with reference to joining the air freshener clip 10 to a louver 54 in a vent opening 52 of an automotive heating and air conditioning vent 50. The air freshener clip 10 may be useful in alternative environments of use, such as attached to ducts for room heating and air conditioning or other areas exposed to air flow, and is not intended to be limited to the environment of use illustrated here. While preferred embodiments of the invention have been described and illustrated here, various changes, substitutions and modifications to the described embodiments will become apparent to those of ordinary skill in the art without thereby departing from the scope and spirit of the invention.

What is claimed is:

1. An air freshener, comprising:
a body with a proximal end and a distal end, and a length and a width, with a width to length ratio of about 1:10 to about 2:6;
a clip arm;
a tip at the distal end of the body; and
an outer portion over-molded over or onto at least a portion of the body, said outer portion formed of polymer material that is loaded with one or more fragrances prior to over-molding.

2. The air freshener of claim 1, wherein over-molding is by a multiple shot molding process.

3. The air freshener of claim 1, wherein over-molding is by injection molding.

4. The air freshener of claim 1, wherein after molding, the outer portion is anchored to the body.

5. The air freshener of claim 1, wherein the clip arm comprises at least one bend formed in the body.

6. The air freshener of claim 1, wherein multiple bends are formed in the clip arm or between the clip arm and the body.

7. The air freshener of claim 1, wherein the clip arm is at the proximal end of the body.

8. The air freshener of claim 1, wherein the body tapers from a first width at its proximal end to a second width at its distal end, with said first width greater than said second width.

9. The air freshener of claim 1, further comprising an upper ridge extending outwardly from a top surface of the body.

10. The air freshener of claim 1, further comprising a lower ridge extending outwardly from a bottom surface of the body.

11. The air freshener of claim 1, further comprising a rib extending outwardly from an exterior surface of the clip arm.

12. The air freshener of claim 1, wherein the body has a concavely curved bottom surface with a radius of curvature in the range of about 1 to about 100 cm.

13. The air freshener of claim 12, wherein the distal end is displaced from a plane defined by an upper exterior surface of the proximal end at an angle in the range of about 1 to about 30 degrees.

14. The air freshener of claim 1, wherein the distal end flexes or bends from a central axis of the body at an angle in the range of from about 1 to about 180 degrees.

15. The air freshener of claim 1, wherein the polymer forming the body is selected from the group consisting of: polyvinylchloride, polyethylene, polyethylene terephthalate (PET), polyoxymethylene (Delrin), polyamide (nylon), low density polyethylene (LDPE), high density polyethylene (HDPE), polypropylene, ethylene vinyl acetate, acrylonitrile butadiene styrene (ABS), styrene-acrylonitrile (SAN), copolymers thereof, and mixtures thereof.

16. The air freshener of claim 1, wherein the polymer forming the outer portion is selected from the group consisting of: polyvinylchloride, polyethylene, low density polyethylene (LDPE), high density polyethylene (HDPE), thermoplastic elastomer (TPE), polypropylene, ethylene vinyl acetate, ELVAX® EVA ethylene vinyl acetate copolymer, acetate, butyrate, propionate, copolymers thereof, and mixtures thereof.

17. The air freshener of claim 1, wherein the fragrance comprises a fragrance material selected from the group consisting of: natural and synthetic fragrance oils, and mixtures thereof.

18. The air freshener of claim 1, further comprising: an accessory removably linked to at least one portion of the body or at least one portion of the clip arm.

19. An air freshener clip for mounting to an air grill having louvers spaced apart to form air passages therebetween for flow of forced air, comprising:
a body with a proximal end and a distal end;
a clip arm adapted for gripping at least one louver;
a tip at the distal end of the body; and
a jacket of polymer material molded over or onto at least a portion of the body, said polymer material loaded with one or more fragrances prior to molding.

20. The air freshener of claim 19, wherein molding is by a multiple shot molding process.

21. The air freshener of claim 19, wherein molding is by injection molding.

22. The air freshener of claim 19, wherein after molding, the jacket is anchored to the body.

23. The air freshener of claim 19, wherein the clip arm comprises at least one bend formed in the body.

24. The air freshener of claim 19, wherein multiple bends are formed in the clip arm or between the clip arm and the body.

25. The air freshener of claim 19, wherein the clip arm is at the proximal end of the body.

26. The air freshener of claim 19, wherein the body tapers from a first width at its proximal end to a second width at its distal end, with said first width greater than said second width.

27. The air freshener clip of claim 19, further comprising an upper ridge extending outwardly from a top surface of the body

28. The air freshener clip of claim 19, further comprising a lower ridge extending outwardly from a bottom surface of the curved body.

29. The air freshener clip of claim 19, wherein the polymer forming the body is selected from the group consisting of: polyvinylchloride, polyethylene, polyethylene terephthalate (PET), polyoxymethylene (Delrin), polyamide (nylon), low density polyethylene (LDPE), high density polyethylene (HDPE), polypropylene, ethylene vinyl acetate, acrylonitrile butadiene styrene (ABS), styrene-acrylonitrile (SAN), copolymers thereof, and mixtures thereof.

30. The air freshener clip of claim 19, wherein the polymer forming the jacket is selected from the group consisting of: polyvinylchloride, polyethylene, low density polyethylene (LDPE), high density polyethylene (HDPE), thermoplastic elastomer (TPE), polypropylene, ethylene vinyl acetate, ELVAX® EVA ethylene vinyl acetate copolymer, acetate, butyrate, propionate, copolymers thereof, and mixtures thereof.

31. The air freshener clip of claim 19, wherein the fragrance comprises a fragrance material selected from the group consisting of: natural and synthetic fragrance oils, and mixtures thereof.

32. The air freshener clip of claim 19, further comprising: an accessory removably linked to the body or to the clip arm.

33. The air freshener clip of claim 19, wherein the body has a length and a width, and the width to length ratio is about 1:10 to about 2:6.

34. The air freshener clip of claim 19, wherein the body has a concavely curved bottom surface with a radius of curvature in the range of about 1 to 100 cm.

35. The air freshener clip of claim 19, wherein the distal end is displaced from a plane defined by an upper exterior surface of the proximal end at an angle in the range of about 1 to about 30 degrees.

36. The air freshener clip of claim 35, wherein the distal end flexes or bends from a plane defined by an upper exterior surface of the proximal end at an angle in the range of from about 1 to about 180 degrees.
